(19) United States
(12) Patent Application Publication
(10) Pub. No.: US 2016/0250496 A1
(43) Pub. Date: Sep. 1, 2016

(54) A SKIN TREATMENT DEVICE FOR LIGHT-BASED TREATMENT OF SKIN TISSUE

(71) Applicant: KONINKLIJKE PHILIPS N.V., Eindhoven (NL)

(72) Inventors: MARTIN JURNA, EINDHOVEN (NL); JONATHAN ALAMBRA PALERO, EINDHOVEN (NL); BABU VARGHESE, EINDHOVEN (NL)

(21) Appl. No.: 15/027,058

(22) PCT Filed: Sep. 23, 2014
(86) PCT No.: PCT/EP2014/070161

§ 371 Date: Apr. 4, 2016

(30) Foreign Application Priority Data
Oct. 4, 2013 (EP) 13187310.1

(51) Int. Cl. A61N 5/06

(52) U.S. Cl. CPC A61N 5/0616; A61N 2005/0632; A61N 2005/0644; A61N 2005/067

(57) ABSTRACT

The invention provides a non-invasive skin treatment device (200) comprising: a light source (10) constructed for emitting treatment light, an optical system (20) constructed for focusing the treatment light to a focus position (340) inside the skin tissue, and a positioning member (50) having a skin contact surface (52), the positioning member (50) and the optical system (20) being displaceable relative to each other for adapting a distance between the skin contact surface (52) and a final lens element (30) of the optical system. The final lens element, in use, faces a skin surface (300), and the positioning member (50) and the optical system are displaceable relative to each other into a treatment position (Pt) of the optical system (20) and into a further position (Pf1) of the optical system (20) different from the treatment position. In use and in the treatment position, the final lens element of the optical system is in contact with the skin surface without an air gap being present between the final lens element and the skin surface.

### FIELD OF THE INVENTION

The invention generally relates to the treatment of skin using light, and more particularly to a skin treatment device having a positioning member defining a distance between the skin surface and an optical system of the skin treatment device.

### BACKGROUND OF THE INVENTION

The desire to maintain a youthful appearance by preventing or reducing wrinkles in the skin is an important issue in human society. Many techniques have been designed to achieve the above issue. One of the techniques, e.g., known from the published international patent application WO 2008/001284 A2, is to create a focal spot in a dermis layer of the skin to be treated. Said WO application discloses a skin treatment device with a laser source and focusing optics. The device emits a laser beam. The power of the laser is selected such that Laser Induced Optical Breakdown (LIOB) affects the skin in order to stimulate re-growth of skin tissue and reduce wrinkles This LIOB is based on strong non-linear absorption of the laser light by the skin tissue, which occurs above a certain threshold value for the power density of the laser light.

Apart from LIOB, also other light-based treatment processes such as selective photothermolysis (e.g. of water), second harmonic generation, third harmonic generation and other higher harmonic generation processes may be used to locally damage tissue in the epidermis or dermis of the skin tissue to stimulate re-growth of the damaged skin tissue to rejuvenate the skin tissue and reduce wrinkles Rejuvenation processes through light-based treatment inside the skin tissue (such as the laser induced optical breakdown) require high intensities in the order of 10¹³ W/cm². Laser sources that are able to generate such high light intensities also may damage, for example, the human eye, and hence often extensive safety measures are required.

In the known treatment device a focal point is created at a fixed treatment depth, somewhere between 0.2 and 2.0 mm. This depth is selected based on the typical composition of human skin. In some cases, however, the optimal treatment depth may be different or treatment may be required at a different or multiple depths to be efficient. The optimal treatment depth depends on, e.g., the thickness of the stratum corneum and the epidermis. The optical system in the known treatment device may comprise an adjustable lens or an adjustable mirror. Both elements, or a combination thereof, may be used to define the focusing action and the treatment depth. However, such adjustable optical elements are relatively expensive.

WO 2009/147617 discloses a light-based skin treatment device including an optical head from which the light exits at a given distance from the skin when the device is placed on the skin. The treatment device includes a gasket of flexible material arraged in such a manner that, when the device is placed against the skin, the gasket is flattened and the optical head can be brought into contact with the skin. Alternatively, the treatment device may also include a movable optical head that is motor driven and that is provided with a system for stopping its movement when the optical head comes into contact with the skin.

### SUMMARY OF THE INVENTION

An object of the invention is to provide a more cost effective non-invasive skin treatment device.

The object is achieved according to the invention by a skin treatment device comprising:

a light source constructed for emitting treatment light,

an optical system constructed for focusing the treatment light to a focus position inside the skin tissue, and

a positioning member having a skin contact surface, the positioning member and the optical system being displaceable relative to each other for adapting a distance between the skin contact surface and a final lens element of the optical system, the final lens element, in use, facing a skin surface, wherein the positioning member and the optical system are displaceable relative to each other into a treatment position of the optical system and into a further position of the optical system different from the treatment position, wherein, in use and in the treatment position, the final lens element of the optical system is in contact with the skin surface without an air gap being present between the final lens element and the skin surface.

The invention is based on the insight that the final lens element has a different optical characteristic when the final lens element is in direct contact with the skin surface, without an air gap between the final lens element and the skin surface, compared to when there is an air gap between the final lens element and the skin surface. By allowing the positioning member and the optical system to be displaceable relative to each other, the skin treatment device according to the invention may have different distances between the final lens element and the skin surface, wherein in the treatment position the final lens element is in contact with the skin surface, thus having a distance substantially equal to zero between the skin surface and the final lens element. Therefore, during the time that the first optical element and the skin surface are in contact with one another, the depth of the focus position inside the skin is well defined to ensure an effective and predefined treatment of the skin tissue. The depth of the focus position in the treatment position is defined by a combination of the optical characteristics of the optical system and the optical characteristics of the skin tissue through which the treatment light is focused towards the focus position inside the skin tissue. By also providing the further position of the positioning member relative to the optical system, the skin treatment device according to the invention is provided with a low-cost additional different functionality.

For example, one of these additional functionalities may be a built-in safety measure in the further position of the optical system, wherein in the further position the distance between the skin contact surface and the optical system may be maximized. In such an arrangement, the positioning member may function as a kind of “cap” to protect the final lens element from scratches. Alternatively, the distance between the skin contact surface and the optical system may be chosen such that any beam of treatment light which is emitted in the further position of the optical system may diverge sufficiently, such that the treatment light is harmless and, for example, can no longer be harmful for the human eye. Alternatively, the additional functionality may be effected such that the further position of the optical system is a further treatment position at a distance from the skin surface, wherein, for example, the treatment light is focused at a different depth inside the skin compared to the treatment position in which the final lens element is in contact with the skin surface. Such additional functionality may make the skin treatment device more versatile and allows the user to apply different treatments and different treatment depths with the same skin treatment device. Although different treatment depths may also be possible with the known treatment device, such known treatment device often requires relatively expensive zoom and focus arrangements, which are not necessary in the skin treatment device according to the invention.

In an embodiment of the skin treatment device, the further position of the optical element is a further treatment position wherein a distance between the final lens element and the skin contact surface is used for focusing the treatment light at a different focus position inside the skin tissue compared to the treatment position. As mentioned before, with the further position being a further treatment position, the skin treatment device according to the invention is allowed to be used for different kinds of skin treatment and is allowed to be used at different skin treatment depths. As a result, the skin treatment device according to the invention is more versatile and more useful to a user. Such a further treatment position typically is a position in which, in use, an air gap is present between the final lens element and the skin surface. The final lens element has to be specifically designed to provide two different focus positions inside the skin tissue, depending on whether the final lens element is in contact with the skin surface or whether there is an air gap between the final lens element and the skin surface. For example, the focusing characteristics of the final lens element may be determined by both an exit surface of the final lens element which, in use, faces the skin tissue and an entrance surface of the final lens element opposite to the exit surface. For example, the entrance surface may be shaped such that, in the treatment position of the optical system, the focus of the treatment light beam is located in a position inside the skin tissue, while the combined optical characteristics of the entrance surface and the exit surface of the final lens element, in the further position of the optical system, may result in a further focus position at the different skin treatment depth inside the skin tissue. In the treatment position of the optical system, the shape of the exit surface of the final lens element may only marginally contribute to the focusing characteristics of the final lens element due to the contact between the exit surface and the skin surface. Especially when an index matching fluid is provided between the exit surface of the final lens element and the skin surface, the change in refractive index between the final lens element and the skin tissue is significantly less compared to the change in refractive index between the final lens element and air, as is the case in the further position of the optical system, which clearly can result in different optical characteristics of the final lens element in the treatment position compared to the further position of the optical system.

In an embodiment of the skin treatment device, the positioning member and the optical system can be mutually locked in the further position of the optical system by means of a locking mechanism. Any locking mechanism may be used to lock the further position of the optical system relative to the positioning member, such as a mechanical pin, a magnetic locking mechanism, a spring locking mechanism, a screwing locking mechanism, and a switch. Such a locking mechanism may provide another cost-effective way to ensure that the focus position inside the skin tissue, when using the skin treatment device in the further position of the optical system, is well defined.

In an embodiment of the skin treatment device, an exit surface of the final lens element has a shape for generating a plurality of focusing positions inside the skin tissue in the further position of the optical system, said exit surface, in use, facing the skin surface, and the final lens element is configured to provide a single focus position inside the skin tissue in the treatment position of the optical system. As indicated before, the change in refractive index between the final lens element and the skin tissue is significantly less compared to the change in refractive index between the final lens element and air, as is the case in the further position of the optical system, which clearly can result in different optical characteristics of the final lens element in the treatment position compared to the further position of the optical system. This change is further reduced when using an index matching fluid. In this embodiment, the light-beam shaping characteristics for creating the plurality of focusing positions inside the skin tissue are substantially completely defined by the exit surface of the final lens element. In the further position of the optical system, the air gap between the exit surface and the skin surface ensures that the exit surface creates the plurality of focus positions inside the skin tissue. In the treatment position of the optical system, the exit surface of the final lens element is in contact with the skin surface, and hence the optical characteristics of the exit surface of the final lens element are substantially annihilated and only the entrance surface of the final lens element determines the focusing process of the light beam. This entrance surface of the final lens element may be constructed so as to provide a single focus position inside the skin tissue in the further position of the optical element.

In an embodiment of the skin treatment device, the final lens element is configured to convert the treatment light into a diverging light beam in the further position of the optical system, and the final lens element is configured to focus the treatment light to the focus position inside the skin tissue in the treatment position of the optical system. This arrangement of the final lens element would result in a “safety-by-design” feature, which results in a strongly diverging light beam when there is no contact between the final lens element and the skin surface. The local intensity of such a strongly diverging light beam would reduce quickly to a level at which the local intensity is too low to be harmful for human skin, and even too low to be harmful to the human eye. Therefore, even when the skin treatment device switches on when not touching the skin surface, the divergence of the light beam of treatment light would result in an additional safety measure.

In a further embodiment of the skin treatment device, an exit surface of the final lens element comprises a concave surface for diverging the treatment light in the further position of the optical system, the exit surface, in use, facing the skin surface. Providing the final lens element with a concave exit surface is one solution to create a strongly diverging light beam when there is no contact between the skin surface and the final lens element. In the treatment position of the optical system in which there is contact between the skin surface and the final lens element, the light-diverging property of the concave exit surface is strongly reduced as explained hereinabove, resulting in a situation in which the remainder of the optical system, apart from the exit surface of the final lens element, determines the focusing characteristics of the treatment light emitted by the skin treatment device according to the invention.

In an alternative embodiment, an exit surface of the final lens element has a conical protruding shape for focusing the treatment light near the exit surface into a diverging annular light beam at a distance from the exit surface, the exit surface in use facing the skin surface. Providing the exit surface of the final lens element with a conical protruding shape is another solution to create a strongly diverging light beam when there is no contact between the skin surface and the final lens element. In the treatment position of the optical system in which there is contact between the skin surface and the final lens element, the light-diverging property of the conically shaped exit surface of the final lens element is strongly reduced as explained hereinabove, resulting in a situation in which the remainder of the optical system, apart from the exit surface of the final lens element, determines the focusing characteristics of the treatment light emitted by the skin treatment device according to the invention. In the further position of the optical system, the conical protruding shape of the exit surface creates a focus very close to the exit surface of the final lens element, after which the beam of treatment light strongly diverges, causing the local light intensity to reduce quickly. Therefore, already at a distance of a few millimeters from the exit surface, the treatment light diverges so strongly that it is harmless, even to the human eye, again creating a “safety-by-design” situation.

In a further embodiment of the skin treatment device, the positioning member comprises a non-transparent wall facing the optical system, the conical protruding shape of the exit surface being configured for diverging the treatment light such that substantially all treatment light, in use, is diverged to impinge on the non-transparent wall of the positioning member in the further position of the optical system. In this embodiment, any light produced by the skin treatment device, with the optical system being in the further position relative to the positioning member, impinges on the non-transparent wall of the skin treatment device and, therefore, is blocked from being uncontrollably emitted from the skin treatment device. Preferably, the non-transparent wall may comprise diffusers to strongly diffuse any light reflecting from that non-transparent wall. Alternatively or additionally, the non-transparent wall may comprise a light absorption material to ensure that the fraction of light reflected from the non-transparent wall is as low as possible.

In a further embodiment of the skin treatment device, an exit surface of the final lens element comprises a Fresnel lens, the exit surface, in use, facing the skin surface. Fresnel lenses are well known in the industry and may have characteristics similar to conventional lenses, but are typically less curved, allowing a better optical contact and coupling with the skin without deforming the skin too much. Using Fresnel lenses also enables the optical system to be produced so as to be relatively compact. Furthermore, when using the Fresnel lens as the exit surface of the final lens element, the optical characteristics of the exit surface may be annihilated when the exit surface is pushed against the skin surface, preferably using an index-matching fluid. The radial step-wise changes of the shape of the exit surface in the Fresnel lens would be easily annihilated, because the air gap between the exit surface and the skin surface would be easily prevented when the Fresnel lens is pressed against the skin surface.

In an embodiment, the optical system is a static optical system without any moving or adjusting lens elements. As indicated before, the use of the positioning member allows the skin treatment device to provide focus positions at different depths inside the skin tissue without the need for any moving or adjusting lens element. Therefore, the overall cost of the skin treatment device according to the invention may be limited, because systems which allow lens elements to move and/or adjust typically impact the price of the device significantly.

In an embodiment of the skin treatment device, a resilient element is arranged between the positioning member and the optical system. Such a resilient element may force the positioning member and the optical element into a predefined mutual position, for example, a position in which the distance between the skin contact surface and the final lens element is maximized. This may be done for safety reasons to protect the final lens element from scratches or to prevent too high intensity light from being emitted from the skin treatment device according to the invention.

In an embodiment of the skin treatment device, the positioning member comprises a through-hole through which the treatment light is focused into the skin tissue, the skin contact surface being arranged on one side of the through-hole. In an embodiment of the skin treatment device, an outer shape of the positioning member is cylindrical, cuboid or triangular prism-shaped. In a further embodiment of the skin treatment device, a skin contact surface of the positioning member is composed of non-corrosive material.

### BRIEF DESCRIPTION OF THE DRAWINGS

FIG. 1 diagrammatically shows an embodiment of a skin treatment device according to the invention,

FIGS. 2A, 2B, 2C and 2D diagrammatically show a first embodiment of a positioning member construction of the skin treatment device according to the invention,

FIGS. 3A and 3B diagrammatically show the first embodiment of the positioning member construction of the skin treatment device according to the invention comprising a locking mechanism,

FIGS. 4A, 4B, 4C and 4D diagrammatically show a second embodiment of a positioning member construction of the skin treatment device according to the invention,

FIGS. 5A and 5B diagrammatically show a third embodiment of a positioning member construction of the skin treatment device according to the invention, and

FIGS. 6A and 6B diagrammatically show an embodiment of a final lens element of a further embodiment of a positioning member construction of the skin treatment device according to the invention.

It should be noted that items which have the same reference numbers in different figures have the same structural features and the same functions, or are the same signals. Where the function and/or structure of such an item have been explained, there is no necessity for repeated explanation thereof in the detailed description.

### DETAILED DESCRIPTION OF EMBODIMENTS

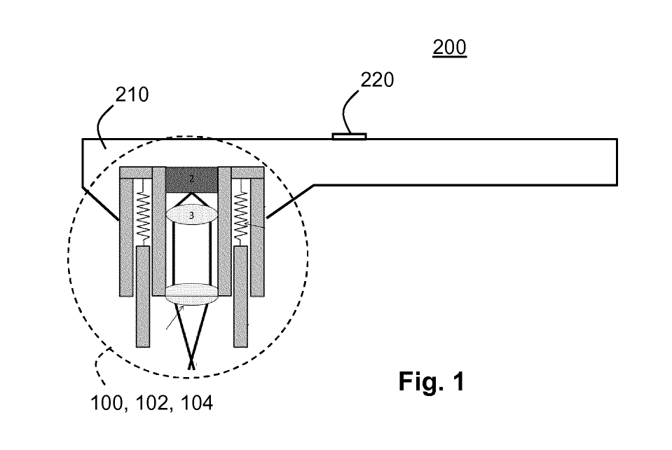

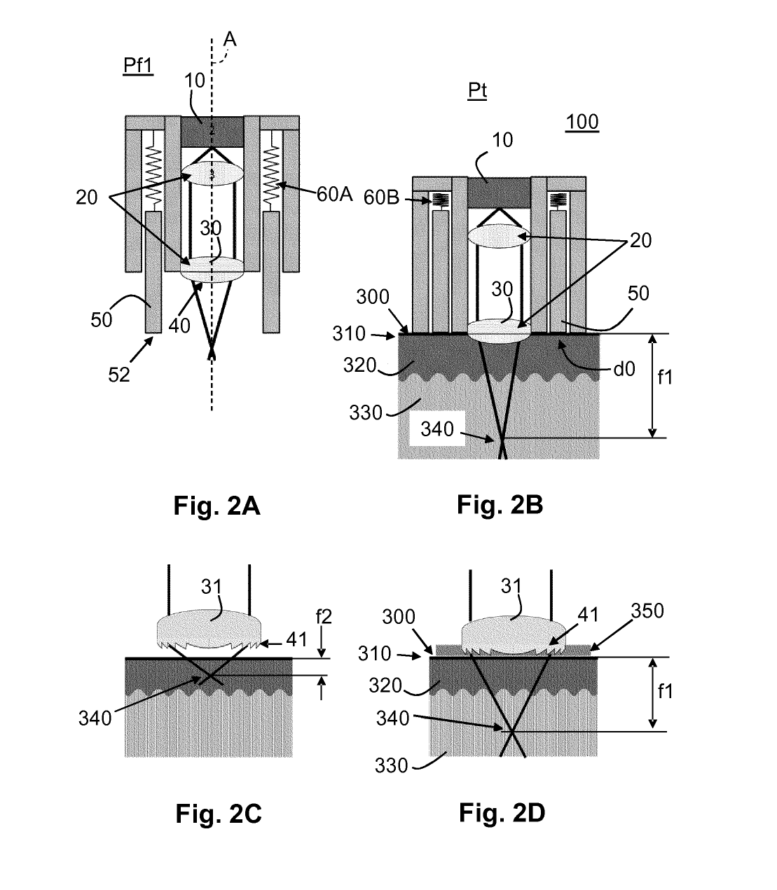

FIG. 1 diagrammatically shows an embodiment of the skin treatment device 200 according to the invention. The skin treatment device 200 according to the invention comprises a housing 210 in which some control circuits (not shown), some connection elements (not shown) and some power means (not shown), such as batteries or a connection to a mains power supply, may be present. Furthermore, a switch 220, such as an on/off switch of the skin treatment device 200, may be present. Also present in the skin treatment device according to the invention is a positioning member construction 100, 102, 104, which comprises at least a part of the optical system 20 (see FIG. 2) and the positioning member 50 (see FIG. 2). The positioning member 50 and the optical system 20 are displaceable relative to each other. In the positioning member construction 100, 102, 104 shown in the FIG. 1 and in the following figures, the positioning member construction 100, 102, 104 also comprises a light source 10 (see FIG. 2). Such a light source 10 may indeed be arranged locally as indicated in the Figures, and may, for example, comprise a laser light source for generating the treatment light. Alternatively, the light source 10 as shown in the Figures may be an output window of a fiber element arranged to transport the treatment light from a remote light source to the optical system 20 such that the optical system 20 can focus the treatment light into the skin tissue, when in use.

FIGS. 2A and 2B diagrammatically show a first embodiment of a positioning member construction 100 of the skin treatment device 200 according to the invention. FIG. 2A shows the first embodiment of the positioning member construction with the optical system 20 in the further position Pf1 (illustrated in FIG. 2A), and FIG. 2B shows the first embodiment of the positioning member construction with the optical system 20 in the treatment position Pt (illustrated in FIG. 2B) in which the final lens element 30 is in contact with the skin surface 300. The positioning member construction 100 shown in FIGS. 2A and 2B comprises a light source 10 and the optical system 20, comprising a plurality of optical elements including the final lens element 30. The positioning member construction 100 comprises the positioning member 50 which is displaceable relative to the optical system 20 in a direction substantially parallel to the optical axis A of the optical system 20. The positioning member 50 comprises a skin contact surface 52 with which the positioning member 50, in use, presses against the skin surface 300 as is shown in FIG. 2B. A resilient element 60A, 60B is shown in FIGS. 2A and 2B, wherein in FIG. 2A the resilient element 60A is shown while no force is applied to the positioning member 50, and in FIG. 2B the resilient element 60B is shown in compressed state due to a force which acts upon the positioning member 50 via the skin contact surface 52 due to the pushing force of the skin treatment device against the skin surface 300 to move the positioning member 50 from the further position Pf1 (of FIG. 2A) to the treatment position Pt (of FIG. 2B). The exit surface 40 is a surface of the final lens element 30 which, in use, faces the skin surface 300.

In FIG. 2B, the first embodiment of the positioning member construction 100 is shown, with the optical system 20 in the treatment position Pt. In the treatment position Pt of the optical system, the positioning member 50 is positioned relative to the optical system 20 such that the final lens element 30 is in direct contact with the skin surface 300. In this arrangement, the contribution of the shape of the exit surface 40 of the final lens element 30 to the optical shaping of the treatment light is strongly reduced and may even be completely annihilated when using index matching fluid, such that the treatment light is focused inside the skin tissue at the focus position 340 at a first focal depth f1. In this arrangement, the distance d0 between the skin contact surface and the final lens element is substantially zero.

The skin comprises multiple layers with different optical properties. The epidermis 320 is composed of the outermost layer. The outermost layer of the epidermis 320 is the stratum corneum 310 which, due to its microscopic fluctuations in roughness, often impedes the coupling of light between the skin treatment device 200 and the skin surface 300. Underneath the epidermis 320, the dermis 330 is situated. The dermis 330 comprises the collagen fibers at which the skin treatment is often aimed. Denaturation of collagen in the dermis layer will trigger the human body to replace the damaged collagen, which will take between one and three months. The new collagen will result in fewer wrinkles and fewer fine lines in the skin. When the cells in the epidermis are damaged, replacement will take several days. This replacement of the epidermal cells will result in a more even skin tone and an overall increase of radiance and glow of a person.

FIGS. 2C and 2D show details of the optical system 20 in which the final lens element 31 is constituted by a Fresnel lens 31. In FIG. 2C, there is an air gap between the exit surface 41 of the final lens element 31 and the skin surface 300 (not indicated in FIG. 2C), which influences the optical characteristics of the overall optical system 20 such that the focus position 340 is located at a second focal depth f2. This may result in a specific distance between the skin contact surface 52 of the positioning member 50 relative to the optical system 20. In the arrangement shown in FIG. 2C, the focus position is located in the epidermis 320 for applying skin treatment to the epidermis. In FIG. 2D, the exit surface 41 is in direct contact with the skin surface 300. Between the exit surface 41 and the skin surface 300, an index-matching fluid 350 is arranged to further improve the optical contact between the exit surface 41 and the skin surface 300. In this arrangement, the optical characteristics of the exit surface 41 are influenced due to the presence of the skin tissue and the index-matching fluid, such that the focus position 340 now is present in the dermis 330, again at the first focal depth f1.

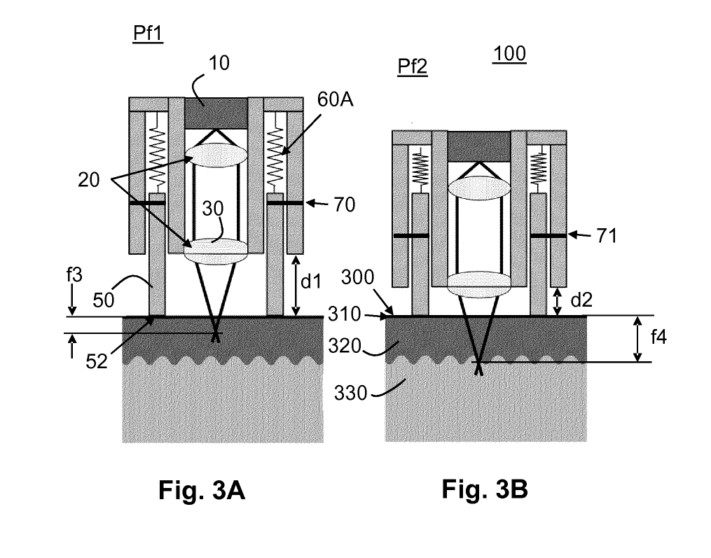

FIGS. 3A and 3B again diagrammatically show the first embodiment of the positioning member construction 100 of the skin treatment device 200 comprising a locking mechanism 70, 71. The positioning member construction 100 shown in FIGS. 3A and 3B again comprises the light source 10, the optical system 20, the positioning member 50 having the skin contact surface 52, the resilient element 60A, and the final lens element 30. In addition to what is shown in FIGS. 2A to 2D, now also the locking mechanism 70, 71 is shown to mutually lock the positioning member 50 and the optical system 20 in the further position Pf1, Pf2. The locking mechanism 70, 71 as shown in FIGS. 3A and 3B is a mechanical pin 70, 71 which locks the position of the positioning member 50 relative to the optical element 30. However, also other locking mechanisms such as a magnetic locking mechanism (not shown), a spring locking mechanism (not shown), a screwing locking mechanism (not shown) or a switch (not shown) may be used without departing from the scope of the invention. In FIG. 3A, the locking mechanism 70 defines a first distance d1 between the skin contact surface 52 and the optical system 20 such that the focal position is located at a third focal depth f3. In the present example, the focal point is located in the epidermis 320 and hence the skin treatment using the skin treatment device 200 with the locking mechanisms 70 is performed in the epidermis 320 of the skin tissue. In FIG. 3B, the locking mechanism 71 defines a second distance d2 between the skin contact surface 52 and the optical system 20 such that the focal point is located deeper into the skin tissue at a fourth focal depth f4, for example, between the epidermis and dermis, aiming at the dermal-epidermal junction. Using such locking mechanisms 70, 71 enables a relatively cost effective way of locking and defining a specific distance d1, d2 between the positioning member 50 and the optical element 20 such that, in use, the focal depth f3, f4 may be defined relatively accurately.

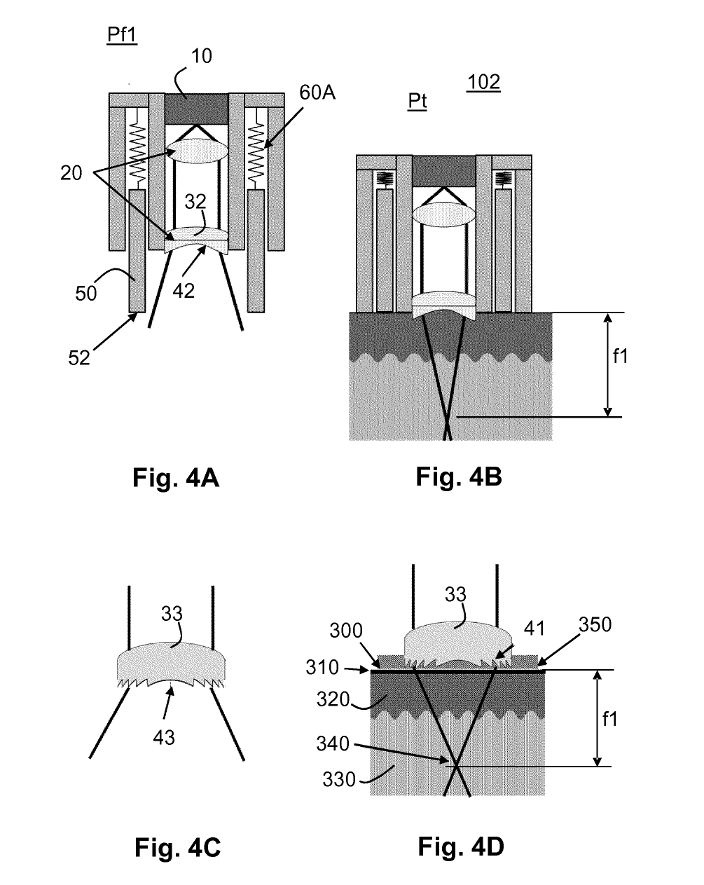

FIGS. 4A, 4B, 4C and 4D diagrammatically show a second embodiment of the positioning member construction 102 of the skin treatment device 200 according to the invention. In FIGS. 4A and 4B, again the light source 10, optical system 20 and positioning member 50 comprising the skin contact surface 52 are shown, together with the resilient element 60A. The optical system 20 comprises optical elements including the final lens element 32 having exit surface 42. In the embodiment of the positioning member construction 102 shown in FIG. 4A, the positioning member 50 is positioned relative to the optical system 20 in the further position Pf1. The exit surface 42 of the final lens element 32 comprises a concave surface 42 which, in the further position Pf1 of the optical system 20, diverges the treatment light in a direction away from the skin treatment device 200. The divergence of the treatment light due to the concave surface 42 preferably is such that, when the diverging light reaches the skin contact surface 52 of the positioning member 50, with the optical system 20 in its further position Pf1, the intensity of the diverging treatment light is such that the diverging treatment light is no longer harmful to the human eye. The resilient element 60A pushes the positioning member 50 relative to the optical system 20 when the skin treatment device 200 is not pushed on to the skin surface 300, such that the concave surface 42 diverges the treatment light as a safety measure. In the situation of FIG. 4B, the skin treatment device 200 is pushed against the skin surface 300, which causes the positioning member 50 and the optical system 20 to be in the treatment position Pt wherein the exit surface 42 is in contact with the skin surface 300, possibly via an index-matching fluid to further improve the optical cooperation between the exit surface 42, or concave surface 42, and the skin surface. As mentioned before, this contact between the exit surface 42 and the skin tissue will significantly reduce the optical contribution of the exit surface 42 to the imaging characteristics of the optical system 20, or will even annihilate this contribution of the exit surface 42, such that the focus position 340 of the treatment light is located at the first focal depth f1.

FIGS. 4C and 4D show details of the optical system 20 in which the final lens element 33 comprises a Fresnel lens 33. As mentioned before, Fresnel lenses 33 are well known in the industry and may have characteristics similar to conventional lenses, but are typically less curved, which allows better optical contact and coupling to the skin surface 300 without deforming the skin surface 300 too much. Using Fresnel lenses 33 also enables the optical system 20 to be more compact. Furthermore, when using the Fresnel lens 33 as the exit surface 43 of the final lens element 33, the optical characteristics of the exit surface 43 may be annihilated when the exit surface 43 is pushed against the skin surface 300, preferably using index-matching fluid 350. The radial step-wise change of the optical shape of the exit surface 43 in the Fresnel lens 33 will be annihilated, because the air gap between the exit surface 43 and the skin surface 300 will be removed when the Fresnel lens 33 is pressed against the skin surface 300. In FIG. 4C, there is an air gap between the exit surface 43 of the final lens element 33 and the skin surface 300 (not shown in FIG. 4C), which influences the optical characteristics of the overall optical system 20 such that the Fresnel lens 33 strongly defocuses the treatment light, as shown in FIG. 4C. In FIG. 4D, the exit surface 43 is in direct contact with the skin surface 300. Between the exit surface 43 and the skin surface 300 an index-matching fluid 350 is provided to further improve the optical contact between the exit surface 43 and the skin surface 300. In this arrangement, the optical characteristics of the exit surface 43 are influenced due to the presence of the skin tissue and the index-matching fluid, such that the optical system 20 now focuses the treatment light towards the focus position 340 in the dermis 330, again at the first focal depth f1.

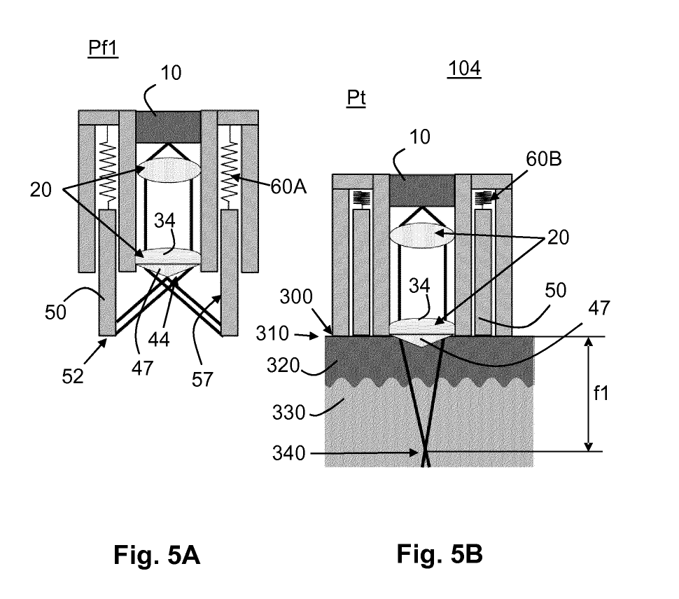

FIGS. 5A and 5B diagrammatically show a third embodiment of the positioning member construction 104 of the skin treatment device 200 according to the invention. In FIGS. 5A and 5B, again the light source 10, optical system 20 and positioning member 50 comprising the skin contact surface 52 are shown, together with the resilient element 60A. The optical system 20 comprises optical elements including the final lens element 34 having the exit surface 44. In the embodiment of the positioning member construction 102 shown in FIG. 5A, the positioning member 50 is positioned relative to the optical system 20 in the further position Pf1. The exit surface 44 of the final lens element 34 comprises a conical protruding shape 47 for focusing the treatment light near the exit surface 44 and creating a diverging annular light beam at a distance from the exit surface 44. Similar to what is shown in FIGS. 4A and 4B, the divergence of the treatment light due to the conical protruding shape 47 preferably is such that when the diverging light reaches the skin contact surface 52 of the positioning member 50, with the positioning member 50 in its further position Pf1, the intensity of the diverging treatment light is such that the diverging treatment light is no longer harmful to the human eye. The resilient element 60A pushes the positioning member 50 relative to the optical system 20 when the skin treatment device 200 is not pushed on to the skin surface 300, such that the conical protruding shape 47 diverges the treatment light as a safety measure. The relative positioning of the positioning member 50 and the optical system 20 in the further position Pf1 may even be such that the diverging treatment light is configured to impinge onto a non-transparent wall 54 of the positioning member 50. This non-transparent wall 54 may be configured and constructed to absorb a substantial part of the impinging treatment light and/or may be configured and constructed to reflect the impinging treatment light diffusely to further reduce the local intensity of the treatment light and further improve the safety of the skin treatment device 200 according to the invention. In the situation of FIG. 5B, the skin treatment device 200 is pushed against the skin surface 300, which causes the positioning member 50 and the optical system 20 to be in the mutual treatment position Pt in which the exit surface 44 is in contact with the skin surface 300, possibly via an index-matching fluid to further improve the optical coupling between the exit surface 44 and the skin surface. As mentioned before, this contact between the exit surface 44 and the skin tissue will significantly reduce the optical contribution of the exit surface 44 to the imaging characteristics of the optical system 20, or will even annihilate this contribution of the exit surface 44, such that the focus position 340 of the treatment light is located at the first focal depth f1.

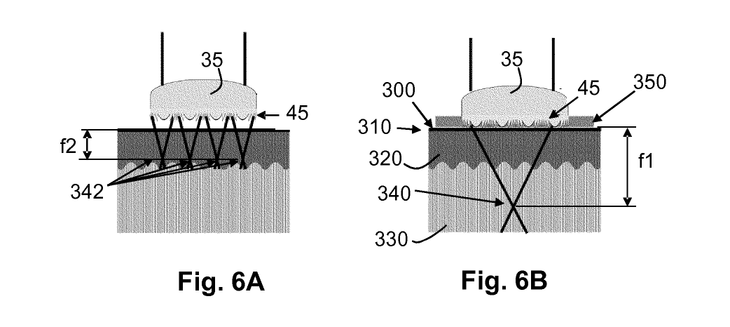

FIGS. 6A and 6B diagrammatically show an embodiment of a final lens element 35 of a further embodiment of the positioning member construction of the skin treatment device 200 according to the invention. In the embodiment of the final lens element 35 shown in FIG. 6A, the exit surface 45 of the final lens element 35 has a shape for generating a plurality of focusing positions 342 inside the skin tissue, with the optical system 20 in its further position Pf1 and with an air gap being present between the exit surface 45 and the skin surface 300. This may be achieved by providing the exit surface 45 with an array of lens elements, each focusing part of the light entering the final lens element 35 into an individual focusing position 342 inside the skin tissue, in the present example at the second focal depth f2 from the skin surface 300 inside the skin tissue. In the situation of FIG. 5B, the exit surface 45 of the skin treatment device 200 according to the invention is pushed against the skin surface 300, which causes the positioning member 50 and the optical system 20 to be in the treatment position Pt in which the exit surface 45 is in contact with the skin surface 300, possibly via an index-matching fluid 350 to further improve the optical coupling between the exit surface 45 and the skin surface 300. As mentioned before, this contact between the exit surface 45 and the skin surface will significantly reduce the optical contribution of the exit surface 45 to the imaging characteristics of the optical system 20, or will even annihilate this contribution of the exit surface 45, such that, in the present situation, the final lens element only provides a single focus point 340 at the first focal depth inside the skin tissue. Therefore, the skin treatment device 200 according to the invention may be used in different modes of operation.

In summary, the invention provides a non-invasive skin treatment device 200 comprising a light source 10 constructed for emitting treatment light, an optical system 20 constructed for focusing the treatment light into a focus position 340 inside the skin tissue, and a positioning member 50 having a skin contact surface 52, the positioning member 50 and the optical system 20 being displaceable relative to each other for adapting and defining a distance between the skin contact surface and a final lens element 30 of the optical system. The final lens element, in use, faces a skin surface 300, and the positioning member and the optical system are displaceable relative to each other into a treatment position Pt and into a further position Pf1 different from the treatment position. In use and in the treatment position of the optical system, the final lens element is in contact with the skin surface for preventing an air gap between the final lens element and the skin surface.

It should be noted that the above-mentioned embodiments illustrate rather than limit the invention, and that those skilled in the art will be able to design many alternative embodiments.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A skin treatment device for light-based treatment of skin tissue, the skin treatment device comprising:
a light source constructed for emitting treatment light,
an optical system constructed for focusing the treatment light to a focus position inside the skin tissue, and
a positioning member having a skin contact surface the positioning member and the optical system being displaceable relative to each other for adapting a distance (d0, d1, d2) between the skin contact surface and a final lens element of the optical system, the final lens element in use facing a skin surface, wherein the positioning member and the optical system are displaceable relative to each other into a treatment position (Pt) of the optical system and into a further position (Pf1, Pf2) of the optical system different from the treatment position (Pt), wherein the skin treatment device is configured to emit, during use, treatment light in the treatment position (Pt) of the optical system and in the further position (Pf1, Pf2) of the optical system, and wherein, in use and in the treatment position (Pt), the final lens element of the optical system is in contact with the skin surface without an air gap being present between the final lens element and the skin surface.

2. The skin treatment device according to claim 1, wherein the further position (Pf1, Pf2) of the optical system is a further treatment position (Pf1, Pf2) wherein a distance (d1, d2) between the final lens element and the skin contact surface is used for focusing the treatment light at a different focus position (f1, f2, f3, f4) inside the skin tissue compared to the treatment position (Pt).

3. The skin treatment device according to claim 1, wherein the positioning member and the optical system can be mutually locked in the further position (Pf1, Pf2) of the optical system by means of a locking mechanisms.

4. The skin treatment device according to claim 17, wherein an exit surface of the final lens element has a shape for generating a plurality of focusing positions) inside the skin tissue in the further position (Pf1, Pf2) of the optical system, the exit surface, in use, facing the skin surface, and wherein the final lens element is configured to provide a single focus position inside the skin tissue in the treatment position (Pt) of the optical system.

5. The skin treatment device according to claim 1, wherein the final lens element is configured to convert the treatment light into a diverging light beam in the further position (Pf1, Pf2) of the optical system, and wherein the final lens element is configured to focus the treatment light to the focus position inside the skin tissue in the treatment position (Pt) of the optical system.

6. The skin treatment device according to claim 5, wherein an exit surface of the final lens element comprises a concave surface for diverging the treatment light in the further position (Pf1, Pf2) of the optical system, the exit surface, in use, facing the skin surface.

7. The skin treatment device according to claim 5, wherein an exit surface of the final lens element has a conical protruding shape for focusing the treatment light near the exit surface into a diverging annular light beam at a distance from the exit surface, the exit surface, in use, facing the skin surface.

8. The skin treatment device according to claim 7, wherein the positioning member comprises a non-transparent wall facing the optical system, the conical protruding shape of the exit surface being configured for diverging the treatment light such that substantially all treatment light, in use, is diverged to impinge on the non-transparent wall of the positioning member in the further position (Pf1, Pf2) of the optical system.

9. The skin treatment device according to claim 1, wherein an exit surface, of the final lens element comprises a Fresnel lens, the exit surface in use, facing the skin surface.

10. The skin treatment device according to claim 1, wherein the optical system is a static optical system without any moving or adjusting lens elements.

11. The skin treatment device according to claim 1, wherein a resilient element is arranged between the positioning member and the optical system.

12. The skin treatment device according to claim 1, wherein the positioning member comprises a through-hole through which the treatment light is focused into the skin tissue, the skin contact surface being arranged on one side of the through-hole.

13. The skin treatment device according to claim 12, wherein an outer shape of the positioning member is cylindrical, cuboid or triangular prism-shaped.

14. The skin treatment device according to claim 1, wherein the skin contact surface of the positioning member is composed of non-corrosive material.
